(19) United States
(12) Patent Application Publication
(10) Pub. No.: US 2021/0304870 A1
(43) Pub. Date: Sep. 30, 2021

(54) DYNAMIC INTELLIGENCE MODULAR SYNTHESIS SESSION GENERATOR FOR MEDITATION

(71) Applicant: MeetKai, Inc., Marina Del Rey, CA (US)

(72) Inventor: James Kaplan, Marina Del Rey, CA (US)

(21) Appl. No.: 17/216,366

(22) Filed: Mar. 29, 2021

Related U.S. Application Data
(60) Provisional application No. 63/000,748, filed on Mar. 27, 2020.

(51) Int. Cl. G16H 20/70; G06N 20/00

(52) U.S. Cl. CPC G16H 20/70; G06N 20/00

(57) ABSTRACT

A system and method for providing a dynamic meditation session to a user where user data is used to generate and output one or more instruction states and one or more non-instruction states. The instruction states include, but are not limited to, audio output, visual output, or both that prompts the user to take a first action or inaction. Feedback data from the user is then used to generate and output an adjusted instruction state and an adjusted non-instruction state to the user. AI processing is used to compare user states or condition, based on biometric feedback, in response to different instruction and non-instructions states to adjust instructions to optimize meditation. The adjusted instruction state includes, but is not limited to, audio output, visual output, or both that prompts the user to take a second action or inaction such that the first action is different than the second action.

### 1. CROSS-REFERENCE TO RELATED APPLICATION

This application claims priority to and incorporates by reference U.S. Provisional Application No. 63/000,748, which was filed on Mar. 27, 2020.

### 2. FIELD OF THE INVENTION

The present invention uses artificial intelligence to assist in meditation and relaxation therapy through a customized Modular Synthesis Session Generator.

### 3. BACKGROUND

Current meditation platforms offer a one size fits all solution. These platforms are available for use via cellphone (iOS and Android), tablet, computers, laptops, and wearable devices. Current platforms would be considered “off the shelf” solutions

These platforms do not allow a user the ability to custom tailor their meditation session. Users are forced to choose a session that cannot be altered before or during the session to custom fit to the user's needs.

An example of a fixed “off the shelf” solution would be the breathing instructions during meditation. In a fixed “off the shelf” solution, every user must breathe at the same rate and pace, every though each user may benefit from have a different pace, rhythm, and flow to their breathing patterns. This solution is not beneficial to the user because it forces a pre-defined meditation instruction on the user does not take into consideration that meditation specifically focuses on the user's body, breathing function, and brain function.

Since these sessions do not allow for a user to give feedback enabling for adaptive dynamic duration control, this reduces meditation benefits when completing a session because the medication was not tailored to their particular needs.

### SUMMARY

The innovation disclosed herein provides a solution to solve the problem of the prior art. This solution offers a customized dynamic session for meditation considering a unique user's meditation needs and their need to naturally let the body, brain, and breathing settle. Each user has a different rate that their body will naturally settle at, and this may change over a course of meditation. If a user does not naturally settle at their proper rate then this can disrupt the body's physical, respiratory, and neural system.

For example, the proposed method and apparatus allows the user the ability for their session to become dynamic. Dynamic means that a user can control their specific session. This innovation is not a “recorded” audio or video session, but instead a dynamic session that builds on itself based on the user's unique profile that takes into consideration historical and real-time data of that specific user. Real time feedback may be used that is obtained from the user to custom tailor the session using artificial intelligence processing.

The dynamic ability of session customization to let the body, breathing, and brain naturally settle through adaptive dynamic duration control is an important key to enabling the meditator to settle down to their proper natural state. An example of the proposed custom session is the ability to control the shortening or extension of the inhale, exhale, or both in the breathing process through our artificial intelligence generator. This is realized through an intelligent algorithm synthesizer (referred to as “IAS”). The IAS is built through a combination of one or more of machine learning, user data, user feedback, and fuzzy logic.

The IAS focuses on two areas of the meditation session. First, an instruction module, which refers to the actual voice command the generator will tell the person. An example of this could be, “focus on your lower back.” Second, a non-instruction module, which refers to the actual amount of time we allow the user to experience the desired command. An example of this could be the sound of a water stream for a dynamically controllable amount of time, 10 seconds.

The solution disclosed herein allows both the instruction module and non-instruction module to be altered through the dynamic synthesis algorithm. Specifically, the solution disclosed herein is a system and method for providing a dynamic meditation session to a user where user data is used to generate and output one or more instruction states and one or more non-instruction states. The instruction states include meditation instructions that may be, but are not limited to audio output, visual output, or both that prompts the user to take a first action or inaction. Feedback data, which may be biometric feedback, from the user is then used to generating and outputting an adjusted instruction state and an adjusted non-instruction state to the user. The adjusted instruction state includes but is not limited to audio output, visual output, or both that prompts the user to take a second action or inaction such that the first action is different than the second action.

In one embodiment, the first set of data is selected from one or more of the following: user account information, user preference, user selection, user input, user biometrics, user history, or auxiliary metadata. The user input may in text format, audio format, image format, or video format. In one embodiment, the feedback data includes but is not limited to user input and/or user biometrics. In one embodiment, a second set of data is used to update the first set of data. The second set of data may include, but is not limited to, user feedback, user preferences, session results, and/or user evaluation of the session. It is contemplated that the analysis of feedback data to generate an adjusted instruction state and an adjusted non-instruction state may include, but is not limited to, comparing a user's current relaxation state in relation to a prior relation state and determining which instruction states or one or more non-instruction states increased the user's relaxation state and responsive to the determining, repeating the instruction states or one or more non-instruction states which increased the user's relation state.

An embodiment of the system includes a user interface configured to receive input and provide instructions to a user, such that the input comprises one or more of the following: user data, non-user data, and feedback data. The embodiment of the system also includes a processor configured to run machine executable code and a memory storing non-transitory machine executable code. The machine executable code is configured to process the user data and non-user data to generate a first instruction state and a first non-instruction state. The instruction state prompts the user to take a first action, which may be achieved through audio output, visual output, or both. The machine executable code is further configured to analyze the feedback data to perform one or more of the following: (1) repeat the first instruction state, (2) repeat the first non-instruction state; (3) adjust the first instruction state; and/or (4) adjust the first non-instruction state. The system may then output the first instruction states, the first non-instruction states, the adjusted first instruction state, and the adjusted first non-instruction state to the user.

It is contemplated that the feedback data includes but is not limited to, user input in text format, audio input, image input, video input, and user biometrics. In one embodiment, the system may adjust the first instruction state by adjusting the output volume of the output of the instruction state, and/or by generating a second instruction state to prompt the user to take a second action or inaction. The first non-instruction state may include one or more of the following: a duration of silence, an audio output, or a visual output. In the same, or another embodiment, the first non-instruction state may be adjusted in one of the following: adjusting the output volume of the first non-instruction state, adjusting the duration of the first non-instruction state, and adjusting the output provided to the user during the first non-instruction state.

One embodiment of the system processes the user data to determining one or more of the following: the user's relaxation state, the user's emotional state, and the user's physical state. The feedback data may be analyzed by comprising comparing a user's current body condition to the user's body condition at a prior point in time. It is contemplated that the machine executable code may use one or more algorithms to process and analyze the user data and the feedback data, and the feedback data may be used to update the one or more algorithm to be executed during the meditation session.

Also disclosed is a method for dynamically adjusting an output in a meditation session, where a first set of data is received from the user, the first set of data indicating a first condition of the user. The first set of data is processed to generate a first instruction output, and the first meditation instruction output is provided to the user. During the presentation of the first output, a second set of data is received from the user, the second set of data obtained from the user and indicating a second condition of the user. The second set of data is compared to the first set of data to determine whether the first instruction output improved the second condition of the user as compared to the first condition of the user, and to determine, responsive to the comparing, either to iterate the first instruction output, or to presenting a second instruction output to the user to improve the meditation session for the user.

In one embodiment, the comparison between the first and second set of data is used to determine whether the first instruction output increased relaxation of the user based on biometric data, and responsive to the first instruction output increasing relaxation of the user, repeating the first instruction output.

It is also contemplated that, responsive to the comparing of the first and second set of data, the method may determine whether to terminate the meditation session.

It is contemplated that the first set of data is selected from one or more of the following: user account information, user preference data, user selection input, user input, user biometrics, user history, and auxiliary metadata. The second set of data may include user input, user biometrics, or both.

### DESCRIPTION OF THE DRAWINGS

The emphasis of the components in the figures are on illustrating the principles of the invention. Thus, the components of the figures are not necessarily to scale. In the figures, like reference numerals designate corresponding parts throughout the different views.

FIG. 1 illustrates an example embodiment of a system for generating and presenting a meditation session.

FIG. 2A illustrates an exemplary timing for the dynamic customization of the duration of instruction states and non-instruction states in a meditation session.

FIG. 2B illustrates another exemplary timing for the dynamic customization of the duration of instruction states and non-instruction states in a meditation session.

FIG. 3A illustrates one exemplary dynamic customization of the content, during a session, of instruction states and non-instruction states in a meditation session.

FIG. 3B another exemplary dynamic customization of the content, during a session, of instruction states and non-instruction states in a meditation session.

FIG. 4 is a flow diagram illustrating how the session generator selects an optimal meditation session based on user information.

FIG. 5 illustrates an example method of generating and presenting a meditation session.

FIG. 6 illustrates an example environment of use of the session generator.

FIG. 7 illustrates a block diagram of an exemplary user device.

FIG. 8 illustrates an example embodiment of a computing device, mobile device, or server in a network environment.

### DESCRIPTION

### Glossary of Terms

AI services: Procedures and methods for a program to accomplish artificial intelligence goals. Examples may include image modelling, text modelling, forecasting, planning, recommendations, search, speech processing, audio processing, audio generation, text generation, image generation, and many more.

Machine learning: a method of data analysis that automates analytical model building. It is a branch of artificial intelligence based on the idea that systems can learn from data, identify patterns and make decisions with minimal human intervention.

Computer logic model (“logic”): program planning tools that define the inputs, outputs, and outcomes of a program in order to explain the thought process behind program design and demonstrate how specific program activities lead to desired results. Examples of logic include standard logic (which applies to concepts that are completely true or completely false, such as 1+1=2) and fuzzy logic (which applies to inherently vague concepts with a degree of truth, such as “this user is calm” with a degree of truth of 0.9).

Fine-tuning/training: an AI service can be “tuned” on a dataset to provide specialized and enhanced capabilities for the specific use case. A model is “trained” with a standard set of data, for instance audio files for word detection. Fine tuning would allow a final step of training for a specific task. For example, where a user speaks defined words, a speech recognition model may be trained using a user's voice and accent.

Meditation: The process of calming or aiding a user's body and mind through breathing patterns.

Real-time: Dynamic and responsive feedback a user receives or provides during meditation.

Session Generator: An algorithm (software, hardware, or both) that utilizes AI services to enable customized meditation with real-time feedback.

Dynamic Intelligence Modular Synthesis Meditation Session (“Meditation Session”): A session generated by the session generator to provide the user with a custom and real-time meditation experience.

Device: Any element running with memory and a CPU and may include a network controller. Optionally, an accelerator can be attached to speed up the computation of AI services.

User Devices: Devices the session generator will run on or communicate with the user, such as smartphones, cell phones, tablet, computer, laptop, television, wearable devices, and webcam devices.

User Information: Data generated by the user or collected from the user before a meditation session, such as user data (for example, account information, location data, user preferences) and user history.

Real-Time User Input: Data generated by the user or collected from the user, including audio recording of the user (such as voice commands or breathing pattern, to respond to user requests or to analyze a user's body condition), image recording of the user (such as a photo of the user to analyze facial expressions or body posture), video recording of the user (to detect and/or analyze the user's movement), biometrics of the user (such as but not limited to heart-rate, oxygen level, blood pressure, or any other metrics that may track a user's body condition).

Auxiliary Metadata: Any data that is not related to the user, such as current date, news, room temperature, weather condition.

Meditation Session Output (“Output”): The session generator may cause a user device to present output responsive to real-time user input. Output may be in the format of dynamic audio, dynamic video, or sound effects. Output may be classified as two types: dynamic instruction output, and dynamic non-instruction output (defined below).

Dynamic Instruction State (“Instruction State”): The session generator may cause a user device to present output responsive to real-time user input. Instruction states is a set of output in the format of dynamic audio, dynamic image, or dynamic video which provide specific guidance to a user in a meditation session. An example of a dynamic audio instruction may be an audio prompt to the user, such as “focus on your lower back”. An example of a dynamic video instruction may be an image of a figure in a suggested meditation post. An example of a dynamic video instruction may be a video showing a figure in a meditation pose, with a glowing indicator on the figure's lower back.

Dynamic Non-Instruction State (“Non-Instruction State”): A set of output that do not provide specific guidance to a user in a meditation session, such as dynamic audio, dynamic video, or silence. An example of a dynamic audio non-instruction may be an audio such as music or various nature sounds (such as ocean waves, rain drops, birds chirping, wind noises, etc.). An example of a dynamic image non-instruction may be the display of a photo of the sunset. An example of a dynamic video non-instruction may be the display of a video recording of waves in the ocean.

As disclosed herein, the innovation introduces a new and improved system to generate dynamic and customized meditation sessions based on user information, real-time user input, and auxiliary metadata. Specifically, an initial meditation session may be generated based on user information and auxiliary metadata. For example, a user may manually input a preference for a stress-relieve meditation session. The stress-relieve meditation session may be further customized based on an analysis of the user's current facial expression or tone of voice to indicate that the user is experiencing a moderate level of stress. The stress-relieve meditation session may be further customized based on an analysis of auxiliary metadata showing it is currently Wednesday, and it is raining outside, and analysis of the user's history indicating the user tends to be more stressed on workdays and dislikes rain, suggesting the user may be experiencing a moderate-to-high level of stress. The initial stress-relieve meditation session may, in response, include lengthy periods of silence to help the user calm down. It is also contemplated that user data, used to custom tailor the meditation session, may include regarding interaction with the artificial intelligence system. For example, a user may perform web searching about any number of topics which can be integrated into the meditation session. These topics include but are not limited to job search, being laid off, vacation, children's issues, death or sickness in the family, promotion, holidays, money issues, sleep issues, anxiety or other mental health issues, moving, graduating, a test or employment review.

The initial stress-relieve meditation session may then be dynamically modified based on real-time user input. For example, three minutes into the meditation session, the user's heart rate or breathing pattern may suggest the user is now experiencing a low level of stress. The modified stress-relieve meditation session may, in response, shorten the period of silence or continue to focus on the aspects of the meditation session which were responsible for reducing the user's perceived stress levels.

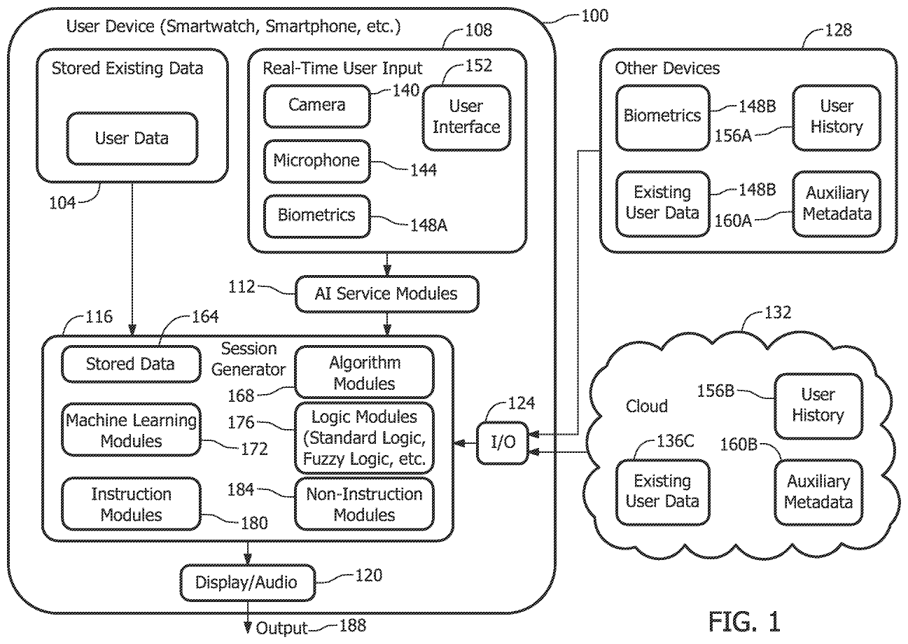

FIG. 1 illustrates an example embodiment of a system for generating and presenting a meditation session. Although described herein as a meditation session, it is contemplated that method and apparatus disclosed herein may be used for any type session that is presented to the user using an artificial intelligence data collection and feedback system. Example of other application beside meditation may include sales training, hypnosis, sleep therapy, waking up sessions, nap sessions, quitting smoking or drug addiction cessation sessions, mental health sessions.

Returning to FIG. 1, user device 100 (such as but not limited to a smartwatch or a smartphone) may include one or more stored data component 104 stored in a memory, a user interface 108, AI service modules 112 stored in a memory to process user input, a session generator 116 stored in memory, various output devices 120 for display output and audio output, and a communication module 124. The communication module 124 may be connected to various other devices 128 and clouds or remote cloud-based servers 132 via any type of electronic connection such as wired networks, wireless networks, optic communication, WiFi, Bluetooth, cellular networks, mesh networks, etc. Many of these elements are software, which may refer to a machine executable code, or data that is stored in memory in a non-transitory state.

The session generator 116 is a software module configured to receive user information and user input from the user device 100, other devices 128, and the cloud 132. Specifically, existing user data 136 may be stored in the stored data component 104 of the user device 100, which the session generator 116 may access. Though not illustrated in FIG. 1, user devices with more room for stored data (such as a smartphone with a large memory capacity) may also store additional user information such as user history and auxiliary metadata. Additional user information and real-time user input 108 may be provided through various hardware such as a camera 140 (for user image input and user video input), microphone 144 (for user audio input), biometrics monitor 148A (such as a smartwatch providing a user's pulse rate, or a smartphone tracking a user's steps taken), and software such as user interface 152 (for a user's text- or touch-based input).

The session generator 116 may access the various input devices 140, 144, 148A, 152 to retrieve user input (which may be monitored by the devices or provided directly by the user). Some user input may require AI service modules 112 to process into another format before the session generator 116 may access and further process the input. For example, when the microphone 144 receives a user's audio command, a speech recognition module may process the audio command into a text-based file, which the session generator 116 may then access and process.

The session generator 116 may also receive information from external sources through the communication module 124. Specifically, the session generator 116 may access real-time user input such as user biometric data from biometric monitors 148B from other devices 128. For example, the session generator may run on a smartphone, but also detect the user's heart rate through a smartwatch that the user is wearing or from one or more devices configured to monitor the user and generate biometric data. The session generator 116 may access user information such as existing user data 126B and user history 156A from other devices 128. For example, the session generator may run on a smartphone, but also access a personal computer that stored the user's account information and a log of the user's heart rate over the past week. The session generator 116 may also access auxiliary metadata 160A from other devices 128. For example, the session generator may run on a smartphone, but also access the room temperature from a smart temperature controller in the same room. Similarly, the session generator 116 may receive, from memory, existing user data 136C, user history 156B, and/or auxiliary metadata 160B from the cloud 132.

The existing user data may include, but is not limited to, user information stored on the user device, which may be user-related data provided by any application installed on the user device such as account information, user preferences, or applications-specific data such as a step counter application providing data on how many steps a user has taken in a day. The user history may include, but is not limited to, past user information such as cookies, browsing history, search history. The biometric data may include, but is not limited to, user-related data on the user's body measurements, such as the heart-rate from a heart-rate monitor. The auxiliary metadata may include, but is not limited to, data not specifically related to the user, such as the date, the weather, news that may be relevant to a zip code identified by the user, etc.

The session generator 116 may store the various information it retrieved as discussed herein in its stored data component 164 (such as a memory). The session generator 116 utilizes algorithm modules 168 to retrieve information from its stored data 164 and analyze the data using machine learning modules 172 and logic modules 176. The session generator 116 then uses the instruction modules 180 and non-instruction modules 184 to generate a meditation session that is customized based on the analyzed user information and data and existing auxiliary metadata 160. The meditation session may be dynamically modified based on real-time user input 140, 144, 148 and real-time auxiliary metadata 160. The session generator 116 may then cause the user device 100 to present the output of the meditation session 188 through its display or audio output devices 120. In various embodiments, the session generator may use anyone, all, or any combination of the above-mentioned data (such as existing user data, user history, user input, user biometrics, auxiliary metadata), as well as additional data not mentioned in FIG. 1, to generate and dynamically modify meditation sessions.

For example, the user device 100 may be a smartphone. The user may use the user interface 152 to input initial user preferences. For example, the user may select a preferred meditation type (such as stress-relieve meditation) or output format (such as audio-only). User preferences may include any of the subsequently discussed variables (such as meditation type, instruction states, and non-instruction states). The stress-relieve meditation session generated based on initial user preference may be a 10-minute meditation session with 10 iterations of one instruction state (such as an audio output of “focus on your breathing”) and 10 iterations of one non-instruction state (such as a 30-second audio file of rain drops).

The session generator 116 may then customize the stress-relieve meditation session based on initial user input by using the camera 140 to take a picture of the user's face. An AI service module 112 capable of analyzing a user's emotions based on facial expression may determine analyze the stress level from the one or more pictures or videos to determine that the user is at a moderate stress level. The session generator 116 may then customize the stress-relieve meditation session to increase the length of the non-instruction states to 35 seconds each. The session generator 116 may analyze the user history 156 to determine that the user dislikes rain or determine from the auxiliary metadata 160 that it is currently raining. The session generator 116 may further customize the stress-relieve meditation session to replace the audio file of rain drops to an audio file of birds chirping. Any combination of instruction or non-instruction can be combined, in any duration, and those factors adjusted based on pre-stored and real-time feedback about the user.

Upon initiation of the meditation session, the session generator 116 may monitor the user's breathing pattern using the microphone 144 or various biometrics input 148. The session generator 116 may determine, 2 minutes into the stress-relief meditation session, that the user's stress level is reduced to low. The session generator 116 may then shorten the remaining iterations of the non-instruction states to 30 seconds each. In another example, the session generator 116 may determine, 2 minutes into the stress-relief meditation session, that the user's stress level continues to rise. The session generator 116 may then alter the non-instruction state to a 35-second period of silence instead.

In one embodiment, the session generator 116 may generate the initial meditation session without any user input of user preferences. In one embodiment, the session generator 116 may rely on only one, or any combination, of user information, user data, and auxiliary metadata to generate and dynamically customize the meditation sessions.

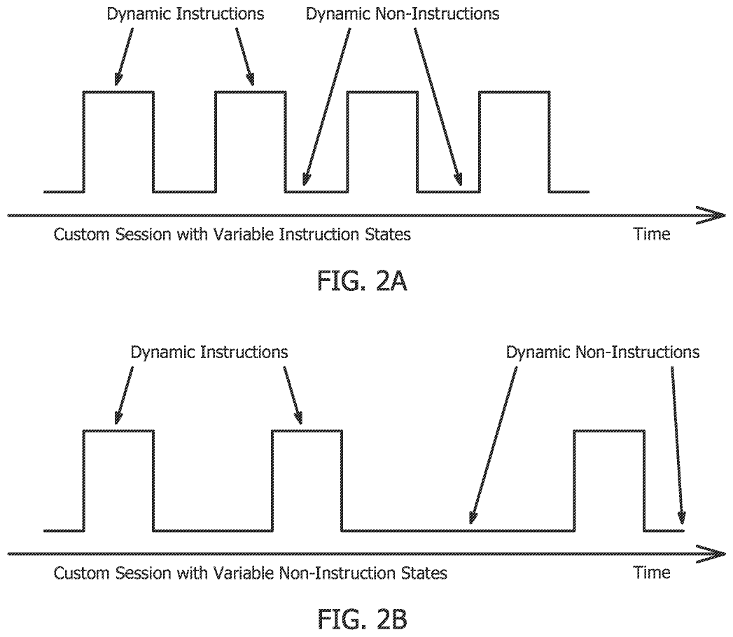

FIGS. 2A and 2B illustrate exemplary timing for the dynamic customization of the duration of instruction states and non-instruction states in a meditation session. Specifically, FIG. 2A illustrates a meditation session where the duration of the instruction and non-instruction states may be consistent over the entire session. For example, all instruction states may be of the same duration. All non-instruction states may also be of the same duration. Further, the duration of instruction states may be the same, or different, as the duration of non-instruction states.

In contrast, FIG. 2B illustrates a meditation session where the instruction states may be of the same duration, while the non-instruction states may vary in duration. For example, the session generator may analyze a user's breathing patterns and determine the user's stress level is rising during a meditation session. The session generator may dynamically increase the duration of the next non-instruction state to facilitate a more rapid reduction of the user's stress level.

FIGS. 2A and 2B are two exemplary examples of meditation sessions. Because meditation sessions are dynamic and customizable based on real-time user input, meditation sessions may include any combination of one or more instruction states and one or more non-instruction states, and each state may vary or be the same in duration. For example, the instruction states may also vary in length based on the user's medication history, such as what results in the best mediation session, or real-time biometric feedback used to adjust duration of the instruction and non-instruction states.

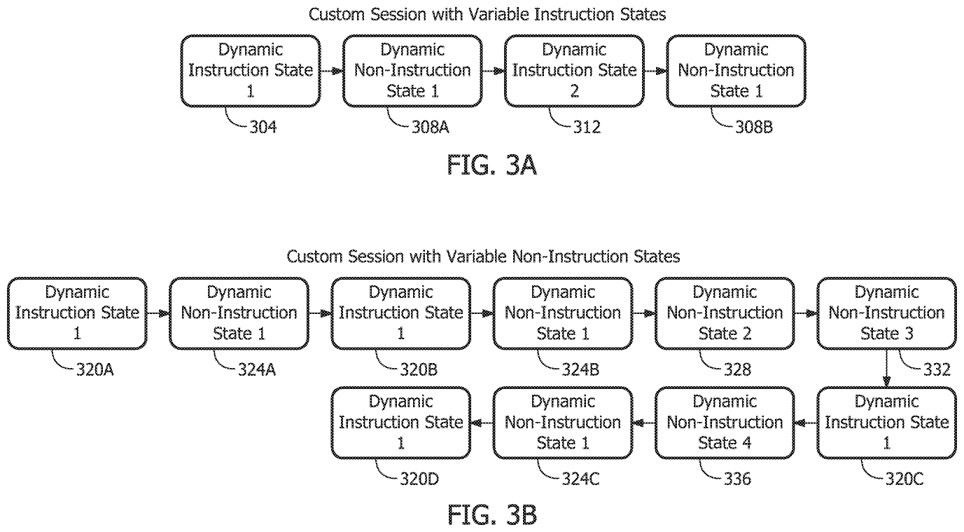

FIGS. 3A and 3B illustrate the dynamic customization of the content, during a session, of instruction states and non-instruction states in a meditation session. FIG. 3A illustrates a meditation session where different instruction states may be dynamically generated, while the same non-instruction state is iterated throughout the meditation session. Specifically, the meditation session may begin with a dynamically generated first instruction state 304, followed by a dynamically generated non-instruction state 308A, followed by a dynamically generated second instruction state 312, and ending with a second iteration of the non-instruction state 308B. For example, during state 308 the session generator may determine from real-time user input that the user's posture has shifted, and the user's stress level is rising, thereby concluding the user's posture is causing stress. Thus, at state 312, the session generator may generate a new instruction state to prompt the user to change posture. On the other hand, the session generator may determine from real-time user input that the non-instruction state used in state 308A remains effective, and thus, should be iterated.

In contrast, FIG. 3B illustrates a meditation session where the same instruction state may be iterated throughout the session, while different non-instruction states may be dynamically generated. Specifically, the session generator may determine the user is at a high level of stress, as indicated by the user's heart rate. The session generator may thus generate a meditation session that may begin with an instruction state 320A that is appropriate for high stress level users, followed by a first non-instruction state 324A tailored as an initial session stage for the user, followed by a second iteration of the instruction state 320B, followed by a second iteration of the non-instruction state 324B. Based on analysis of real-time user input (biometric and other type input), the session generator may then determine that additional and different non-instruction states are needed (for example, based on determination that the user's stress level remains high), and thus output a second non-instruction state 328 that may be specifically designed to initiate relaxation or meet another medication goal. Based on analysis of further real-time user input, the session generator may determine that the second non-instruction state 328 has not achieved the desired effect (such as the stress level reducing from high to medium). Thus, the session generator may attempt a third non-instruction state 332. Upon achieving the desired effect, the session generator may then output the next iteration of the instruction state 320C, followed by a fourth non-instruction state 336 appropriated for the user's current state (such as a non-instruction state appropriate for medium stress level users). Upon detecting a further reduction of the user's stress level from medium to low, the session generator may then output a second iteration of the generic first non-instruction state 324C again, followed by a final iteration of the instruction state 320D to end the meditation session.

As can be seen, the type of non-instructions states can vary. For example, if classical music is not relaxing the user, then a different non-instruction state may be provided such as silence, or the sound of rain fall. Types of non-instructions may occur other than music, such as lighting, massage control, or other features.

FIGS. 3A and 3B are two exemplary examples of meditation sessions. Because meditation sessions are dynamic and customizable based on real-time user input (feedback), meditation sessions may include any combination of one or more instruction states and one or more non-instruction states, and each state may vary or be the same in the content of its output. These instruction states and non-instruction states may also vary in duration, as discussed above.

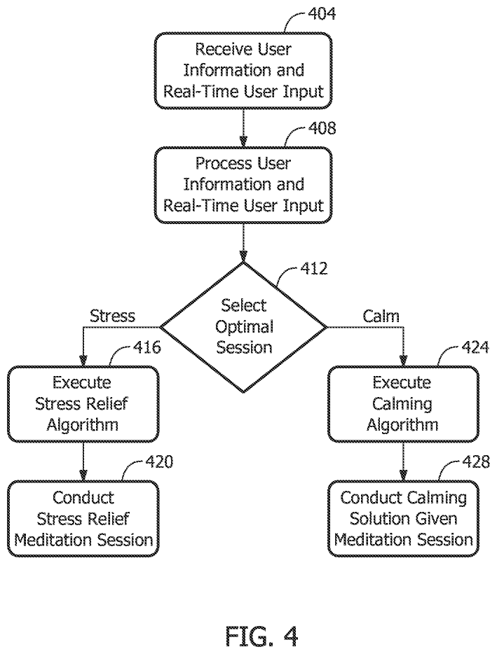

FIG. 4 is a flow diagram illustrating how the session generator selects an optimal meditation session based on user information. At a step 404, the session generator receives stored user information and real-time user input (user input and biometric feedback) using the various systems and methods described in FIG. 1. At a step 408, the session generator processes the received user information and real-time user input using its machine learning and logic modules to determine the user condition. The user condition represents the state of the user, such as stressful, worried, tired, sore, and the reasons for the user's condition. The data collected from the user is used to determine their condition. By way of example, the user may tell the session generator that they are worried about work and not sleeping well. The session generator can collect biometric feedback from the user to supplement the model of the user's condition. The session generator may also use prior data regarding the user to further supplement the model of the user's current condition. For example, the session generator may access the subject matter the user has been searching on the web and activities the user has been doing recently.

At a step 412, the session generator selects and customizes a meditation session customized to the user condition. Further customization occurs during the session. For example, the session generator may compare the real-time input of the user's heart rate to the average heart rate in the user history to determine that the user's heart rate is currently elevated. As a result, the session generator may determine the user condition is stress. The session generator may then, at a step 416, execute the stress relief algorithm and generate a meditation session using the instruction modules and the non-instruction modules related to stress relief. As part of generating a customized meditation session, the session generator may analyze prior meditation sessions or history of meditation session results. Then at a step 420, the session generator may conduct the customized stress relief meditation session by outputting the customized instruction and non-instruction states.

As another example, the session generator may analyze a real-time user input in the form of a video feed of the user's current facial expression. The session generator may determine the user condition is calm. The session generator then, at a step 424, executes a calming algorithm and generate a meditation session using the instruction modules and the non-instruction modules related to calming. Then, at a step 428, the session generator may conduct the customized calming session or stress relief meditation session by outputting the customized instruction and non-instruction states.

FIG. 4 presents two of many examples of possible user conditions, and possible meditation sessions responsive to the user condition. It is contemplated that a wide range of user condition may be detected (such as anger, anxiety, excitement, tension, tiredness, life events, types of worries, medical situations/conditions etc.) and an exponential amount of customizable meditation sessions may be generated using a varying number and variety of instruction states and non-instruction states.

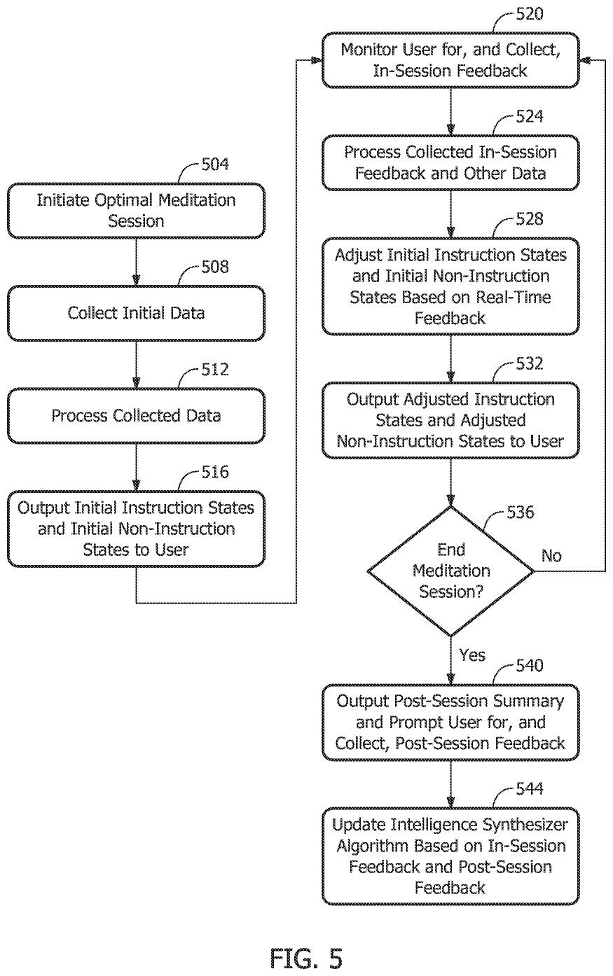

FIG. 5 illustrates a flow diagram of an example method of generating and presenting a meditation session, and how individual instruction states and non-instruction states may be optimized based on real-time user input. This method may use AI services, machine learning, and model fine-tuning. At a step 504, the optimal meditation session may be initiated based on user information. The optimal meditation session may be selected automatically by the session generator (such as based on user preferences and user history), or a user may select a desired meditation session manually. At a step 508, the session generator may collect real-time user input using the various methods discussed in FIG. 1. At a step 512, the session generator may analyze the collected real-time user input to identify the user's initial condition. The analysis may include comparing the user's condition and needs to mediation instructions, states, and types of sessions which are known or predicted to best aid the user.

At a step 516, the session generator, based on the user's initial condition, generates and outputs initial instruction states and non-instruction states customized to the user's initial condition. For example, a user may have initially selected a stress-relief meditation session. The session generator may, based on real-time user input of the user's heart rate, determine the user's current stress level is moderate-to-high. The session generator may, in response, output stress-related initial instruction states and non-instruction states customized to a moderate-to-high level of stress. Alternatively, the session generator may, based on an analysis of the user input, user history, and user biometrics suggest or propose a different type mediation session that initially selected by the user to provide a more helpful session to the user.

At a step 520, the session generator may continue to monitor for real-time user input and collect such user input. At a step 524, the session generator may process the collected real-time input to determine the updated user condition during the meditation session. The term ‘real-time’ input may include but is not limited to user biometric data and user input. At a step 528, the session generator may adjust the instruction states and non-instruction states based on the updated user condition to tailor the session to maximize the helpful effects of the meditation.

For example, during the stress-relief meditation session, the session generator may determine the user's stress level has dropped to a medium level, then to a low level. The session generator may, in response, output adjusted instruction states and non-instruction states customized to a medium level of stress, then customized to a low level of stress. Similarly, the session generator records and stores the type of session and session event which caused the user's perceived stress level to drop so that those same sessions and events for future use. Aspects of session which showed not beneficial effect are also noted so as to possibly be avoided in the future.

At a step 536, the session generator may determine whether the meditation session may end. The meditation session may end based on user information (such as a user preference indicating a desired duration for the meditation session), real-time user input (such as the user's voice-command “end meditation session”), or analysis based on real-time user input (such as determination that a user's stress level is reduced to a low level during a stress relief meditations session). If the meditation session does not end, then steps 520-528 may be repeated throughout the meditation session.

If, on the other hand, the session generator determines the meditation session may end, then the session generator may output customized end-of session instruction states and non-instruction states. In a step 540, upon conclusion of the meditation session, the session generator may also output post-session summaries (such as number values, visual representations, and analysis of the real-time user input collected). The session generator may also prompt the user for additional feedback. For example, at the conclusion of a stress-relief meditation session, the session generator may output a list of the user's heart-rate collected at intervals, and an analysis showing the user's gradual reduction of stress level from high to low. The session generator may also prompt the user to rate the effectiveness of the meditation session, and the user's own evaluation of stress level at the conclusion of the meditation session.

At a step 544, the machine learning modules in the session generator may use the real-time user input collected during the meditation session and the post-session feedback to train and fine-tune the logic and algorithm modules. For example, where the session generator determined the user was at a low stress level based on a heart rate of 70 bpm at the conclusion of the meditation session, but the user rated his stress level at medium, the session generator may update its logic and algorithm modules to associate a user's heart rate of 70 bpm with medium stress levels instead of low. Similarly, the successfulness (and aspects which caused the success) and user feedback of the session are recorded for future use to custom tailor future sessions, along with real-time user feedback.

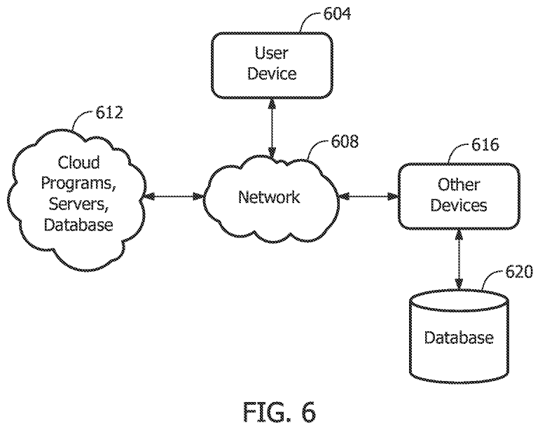

FIG. 6 illustrates an example environment of use of the session generator. In FIG. 6, the session generator may be an application installed on a user device 604. The user device 604 may be connected to cloud programs, servers, and/or databases 612 and other devices 616 via a network 608 such as a LAN, WAN, PAN, or the Internet. Other devices 616 may be connected to their own databases 620. The session generator may thus access resources from all connected programs, devices, servers, and/or databases.

For example, the session generator may be an application installed on a user's smartphone. The session generator may use auxiliary metadata from a connected cloud server, or a heart rate monitor on a connected smartwatch to customize the user's meditation session.

FIG. 6 is only one example environment. It is contemplated that the session generator may also be stored in a cloud or on other devices, which a user device may access remotely via any type of electronic connection such as wired networks, wireless networks, optic communication, WiFi, Bluetooth, cellular networks, mesh networks, etc.

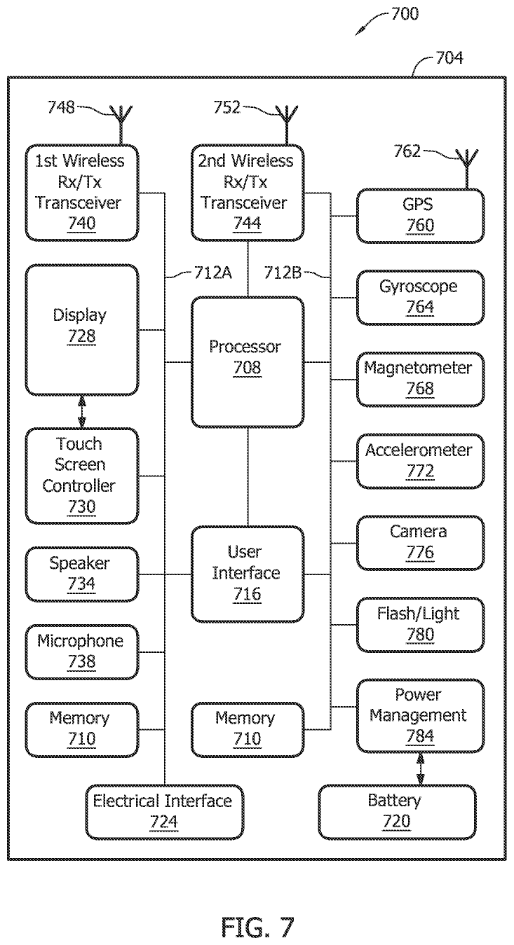

FIG. 7 illustrates an example embodiment of a mobile device on which a solution generator may operate, also referred to as a user device which may or may not be mobile. This is but one possible mobile device configuration and as such, it is contemplated that one of ordinary skill in the art may differently configure the mobile device. The mobile device 700 may comprise any type of mobile communication device capable of performing as described below. The mobile device may comprise a Personal Digital Assistant (“PDA”), cellular telephone, smart phone, tablet PC, wireless electronic pad, an IoT device, a “wearable” electronic device or any other computing device.

In this example embodiment, the mobile device 1300 is configured with an outer housing 704 configured to protect and contain the components described below. Within the housing 704 is a processor 708 and a first and second bus 712A, 712B (collectively 712). The processor 708 communicates over the buses 712 with the other components of the mobile device 700. The processor 708 may comprise any type processor or controller capable of performing as described herein. The processor 708 may comprise a general purpose processor, ASIC, ARM, DSP, controller, or any other type processing device. The processor 708 and other elements of the mobile device 700 receive power from a battery 720 or other power source. An electrical interface 724 provides one or more electrical ports to electrically interface with the mobile device, such as with a second electronic device, computer, a medical device, or a power supply/charging device. The interface 724 may comprise any type electrical interface or connector format.

One or more memories 710 are part of the mobile device 700 for storage of machine readable code for execution on the processor 708 and for storage of data, such as image data, audio data, user data, location data, accelerometer data, or any other type of data. The memory 710 may comprise RAM, ROM, flash memory, optical memory, or micro-drive memory. The machine readable code (software modules and/or routines) as described herein is non-transitory.

As part of this embodiment, the processor 708 connects to a user interface 716. The user interface 716 may comprise any system or device configured to accept user input to control the mobile device. The user interface 716 may comprise one or more of the following: microphone, keyboard, roller ball, buttons, wheels, pointer key, touch pad, and touch screen. A touch screen controller 1330 is also provided which interfaces through the bus 712 and connects to a display 728.

The display comprises any type display screen configured to display visual information to the user. The screen may comprise a LED, LCD, thin film transistor screen, OEL CSTN (color super twisted nematic), TFT (thin film transistor), TFD (thin film diode), OLED (organic light-emitting diode), AMOLED display (active-matrix organic light-emitting diode), capacitive touch screen, resistive touch screen or any combination of these technologies. The display 728 receives signals from the processor 708 and these signals are translated by the display into text and images as is understood in the art. The display 728 may further comprise a display processor (not shown) or controller that interfaces with the processor 708. The touch screen controller 730 may comprise a module configured to receive signals from a touch screen which is overlaid on the display 728.

Also part of this exemplary mobile device is a speaker 734 and microphone 738. The speaker 734 and microphone 738 may be controlled by the processor 708. The microphone 738 is configured to receive and convert audio signals to electrical signals based on processor 708 control. Likewise, the processor 708 may activate the speaker 734 to generate audio signals. These devices operate as is understood in the art and as such are not described in detail herein.

Also connected to one or more of the buses 712 is a first wireless transceiver 740 and a second wireless transceiver 744, each of which connect to respective antennas 748, 752. The first and second transceiver 740, 744 are configured to receive incoming signals from a remote transmitter and perform analog front-end processing on the signals to generate analog baseband signals. The incoming signal may be further processed by conversion to a digital format, such as by an analog to digital converter, for subsequent processing by the processor 708. Likewise, the first and second transceiver 740, 744 are configured to receive outgoing signals from the processor 708, or another component of the mobile device 708, and up convert these signals from baseband to RF frequency for transmission over the respective antenna 748, 752. Although shown with a first wireless transceiver 740 and a second wireless transceiver 744, it is contemplated that the mobile device 700 may have only one such system or two or more transceivers. For example, some devices are tri-band or quad-band capable, or have Bluetooth®, NFC, or other communication capability.

It is contemplated that the mobile device, and hence the first wireless transceiver 740 and a second wireless transceiver 744 may be configured to operate according to any presently existing or future developed wireless standard including, but not limited to, Bluetooth, WI-FI such as IEE 802.11 a,b,g,n, wireless LAN, WMAN, broadband fixed access, WiMAX, any cellular technology including CDMA, GSM, EDGE, 3G, 4G, 5G, TDMA, AMPS, FRS, GMRS, citizen band radio, VHF, AM, FM, and wireless USB.

Also part of the mobile device is one or more systems connected to the second bus 712B which also interface with the processor 708. These devices include a global positioning system (GPS) module 760 with associated antenna 762. The GPS module 760 is capable of receiving and processing signals from satellites or other transponders to generate location data regarding the location, direction of travel, and speed of the GPS module 760. GPS is generally understood in the art and hence not described in detail herein. A gyroscope 764 connects to the bus 712B to generate and provide orientation data regarding the orientation of the mobile device 704. A magnetometer 768 is provided to provide directional information to the mobile device 704. An accelerometer 772 connects to the bus 712B to provide information or data regarding shocks or forces experienced by the mobile device. In one configuration, the accelerometer 772 and gyroscope 764 generate and provide data to the processor 708 to indicate a movement path and orientation of the mobile device.

One or more cameras (still, video, or both) 776 are provided to capture image data for storage in the memory 710 and/or for possible transmission over a wireless or wired link, or for viewing at a later time. The one or more cameras 776 may be configured to detect an image using visible light and/or near-infrared light. The cameras 776 may also be configured to utilize image intensification, active illumination, or thermal vision to obtain images in dark environments. The processor 708 may process machine readable code that is stored on the memory to perform the functions described herein.

A flasher and/or flashlight 780, such as an LED light, are provided and are processor controllable. The flasher or flashlight 780 may serve as a strobe or traditional flashlight. The flasher or flashlight 780 may also be configured to emit near-infrared light. A power management module 784 interfaces with or monitors the battery 720 to manage power consumption, control battery charging, and provide supply voltages to the various devices which may require different power requirements.

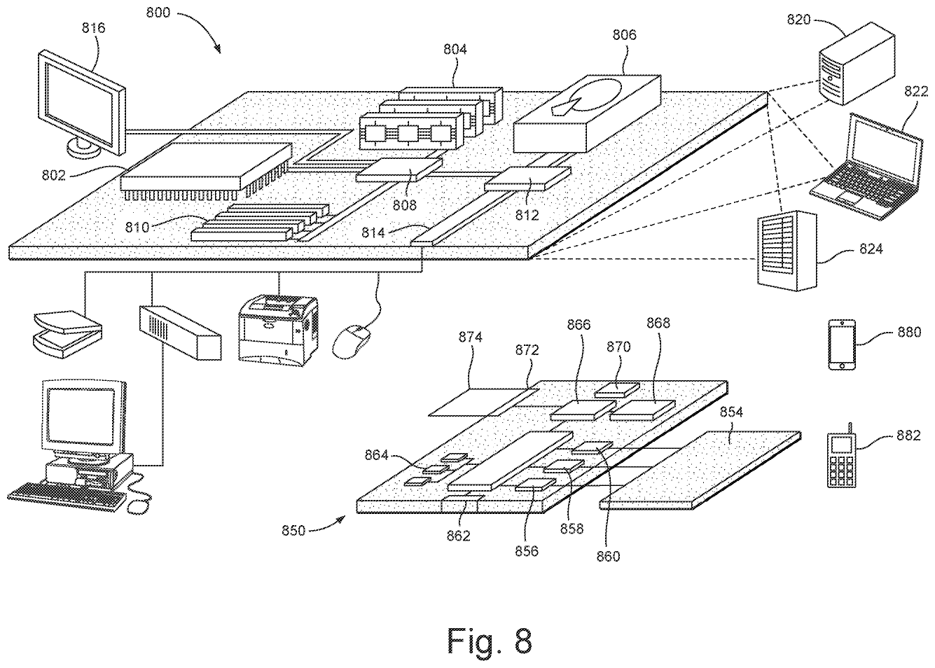

FIG. 8 is a schematic of a computing or mobile device, or server, such as one of the devices described above, according to one exemplary embodiment. Computing device 800 is intended to represent various forms of digital computers, such as smartphones, tablets, kiosks, laptops, desktops, workstations, personal digital assistants, servers, blade servers, mainframes, and other appropriate computers. Computing device 850 is intended to represent various forms of mobile devices, such as personal digital assistants, cellular telephones, smart phones, and other similar computing devices. The components shown here, their connections and relationships, and their functions, are meant to be exemplary only, and are not meant to limit the implementations described and/or claimed in this document.

Computing device 800 includes a processor 802, memory 804, a storage device 806, a high-speed interface or controller 808 connecting to memory 804 and high-speed expansion ports 810, and a low-speed interface or controller 812 connecting to low-speed bus 814 and storage device 806. Each of the components 802, 804, 806, 808, 810, and 812, are interconnected using various busses, and may be mounted on a common motherboard or in other manners as appropriate. The processor 802 can process instructions for execution within the computing device 800, including instructions stored in the memory 804 or on the storage device 806 to display graphical information for a GUI on an external input/output device, such as display 816 coupled to high-speed controller 808. In other implementations, multiple processors and/or multiple buses may be used, as appropriate, along with multiple memories and types of memory. Also, multiple computing devices 800 may be connected, with each device providing portions of the necessary operations (e.g., as a server bank, a group of blade servers, or a multi-processor system).

The memory 804 stores information within the computing device 800. In one implementation, the memory 804 is a volatile memory unit or units. In another implementation, the memory 804 is a non-volatile memory unit or units. The memory 804 may also be another form of computer-readable medium, such as a magnetic or optical disk.

The storage device 806 is capable of providing mass storage for the computing device 800. In one implementation, the storage device 806 may be or contain a computer-readable medium, such as a hard disk device, an optical disk device, or a tape device, a flash memory or other similar solid-state memory device, or an array of devices, including devices in a storage area network or other configurations. A computer program product can be tangibly embodied in an information carrier. The computer program product may also contain instructions that, when executed, perform one or more methods, such as those described above. The information carrier is a computer- or machine-readable medium, such as the memory 804, the storage device 806, or memory on processor 802.

The high-speed controller 808 manages bandwidth-intensive operations for the computing device 800, while the low-speed controller 812 manages lower bandwidth-intensive operations. Such allocation of functions is exemplary only. In one implementation, the high-speed controller 808 is coupled to memory 804, display 816 (e.g., through a graphics processor or accelerator), and to high-speed expansion ports 810, which may accept various expansion cards (not shown). In the implementation, low-speed controller 812 is coupled to storage device 806 and low-speed bus 814. The low-speed bus 814, which may include various communication ports (e.g., USB, Bluetooth, Ethernet, wireless Ethernet) may be coupled to one or more input/output devices, such as a keyboard, a pointing device, a scanner, or a networking device such as a switch or router, e.g., through a network adapter.

The computing device 800 may be implemented in a number of different forms, as shown in the figure. For example, it may be implemented as a standard server 820, or multiple times in a group of such servers. It may also be implemented as part of a rack server system 824. In addition, it may be implemented in a personal computer such as a laptop computer 822. Alternatively, components from computing device 800 may be combined with other components in a mobile device (not shown), such as device 850. Each of such devices may contain one or more of computing device 800, 850, and an entire system may be made up of multiple computing devices 800, 850 communicating with each other.

Computing device 850 includes a processor 852, memory 864, an input/output device such as a display 854, a communication interface 866, and a transceiver 868, among other components. The computing device 850 may also be provided with a storage device, such as a micro-drive or other device, to provide additional storage. Each of the components 852, 864, 854, 866, and 868, are interconnected using various buses, and several of the components may be mounted on a common motherboard or in other manners as appropriate.

The processor 852 can execute instructions within the computing device 850, including instructions stored in the memory 864. The processor may be implemented as a chipset of chips that include separate and multiple analog and digital processors. The processor may provide, for example, for coordination of the other components of the computing device 850, such as control of user interfaces, applications run by the computing device 850, and wireless communication by the computing device 850.

Processor 852 may communicate with a user through control interface 858 and display interface 856 coupled to a display 854. The display 854 may be, for example, a TFT LCD (Thin-Film-Transistor Liquid Crystal Display) or an OLED (Organic Light Emitting Diode) display, or other appropriate display technology. The display interface 856 may comprise appropriate circuitry for driving the display 854 to present graphical and other information to a user. The control interface 858 may receive commands from a user and convert them for submission to the processor 852. In addition, an external interface 862 may be provided in communication with processor 852, to enable near area communication of device 850 with other devices. External interface 862 may provide, for example, for wired communication in some implementations, or for wireless communication in other implementations, and multiple interfaces may also be used.

The memory 864 stores information within the computing device 850. The memory 864 can be implemented as one or more of a computer-readable medium or media, a volatile memory unit or units, or a non-volatile memory unit or units. Expansion memory 874 may also be provided and connected to the computing device 850 through expansion interface 872, which may include, for example, a SIMM (Single In Line Memory Module) card interface. Such expansion memory 874 may provide extra storage space for the computing device 850 or may also store applications or other information for the computing device 850. Specifically, expansion memory 874 may include instructions to carry out or supplement the processes described above and may include secure information also. Thus, for example, expansion memory 874 may be provided as a security module for the computing device 850 and may be programmed with instructions that permit secure use of the computing device 850. In addition, secure applications may be provided via the SIMM cards, along with additional information, such as placing identifying information on the SIMM card in a non-hackable manner.

The memory may include for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 864, expansion memory 874, or memory on processor 852, that may be received for example, over transceiver 868 or external interface 862.

The computing device 850 may communicate wirelessly through communication interface 866, which may include digital signal processing circuitry where necessary. Communication interface 866 may provide for communications under various modes or protocols, such as GSM voice calls, SMS, EMS, or MMS messaging, CDMA, TDMA, PDC, WCDMA, CDMA2000, or GPRS, among others. Such communication may occur for example, through a radio-frequency transceiver 868. In addition, short-range communication may occur, such as using a Bluetooth, Wifi, or other such transceiver (not shown). In addition, GPS (Global Positioning system) receiver module 870 may provide additional navigation- and location-related wireless data to the computing device 850, which may be used as appropriate by applications running on the computing device 850.

The computing device 850 may also communicate audibly using audio codec 860, which may receive spoken information from a user and convert it to usable digital information. Audio codec 860 may likewise generate audible sound for a user, such as through a speaker, e.g., in a handset of the computing device 850. Such sound may include sound from voice telephone calls, may include recorded sound (e.g., voice messages, music files, etc.) and may also include sound generated by applications operating on the computing device 850.

The computing device 850 may be implemented in a number of different forms, as shown in the figure. For example, it may be implemented as a cellular telephone 860. It may also be implemented as part of a smart phone 882, personal digital assistant, a computer tablet, or other similar mobile device.

Thus, various implementations of the systems and techniques described here can be realized in digital electronic circuitry, integrated circuitry, especially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor and can be implemented in a high-level procedural and/or object-oriented programming language, and/or in assembly/machine language. As used herein, the terms “machine-readable medium” and “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device (e.g., CRT (cathode ray tube) or LCD (liquid crystal display) monitor) for displaying information to the user, a keyboard, and a pointing device (e.g., mouse, joystick, trackball, or similar device) by which the user can provide input to the computer. Other kinds of devices can be used to provide for interaction with a user as well, for example; feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system (e.g., computing device 800 and/or 850) that includes a back end component (e.g., data server, slot accounting system, player tracking system, or similar), or that includes a middleware component (e.g., application server), or that includes a front-end component such as a client computer having a graphical user interface or a Web browser through which a user can interact with an implementation of the systems and techniques described here, or any combination of such back-end, middleware, or front-end components. The components of the system can be interconnected by any form or medium of digital data communication, such as a communication network. Examples of communication networks include a local area network (“LAN”), a wide area network (“WAN”), and the Internet.

The computing system can include clients and servers. A client and server are generally remote from each other and typically interact through a communication network. The relationship of client and server arises by virtue of computer programs running on the respective computers and having a client-server relationship to each other.

While various embodiments of the invention have been described, it will be apparent to those of ordinary skill in the art that many more embodiments and implementations are possible that are within the scope of this invention. In addition, the various features, elements, and embodiments described herein may be claimed or combined in any combination or arrangement.

What is claimed is:

1. A method for providing a dynamic meditation session to a user comprising:
receiving input from a user to initiate a meditation session;
retrieving a first set of user data, the first set of user data comprising data regarding one or more aspects of the user;
processing the first set of data to generate and output to the user one or more instruction states and one or more non-instruction state, the instruction states comprising audio output, visual output, or both that prompts the user to take a first action or inaction;
receiving feedback data from the user;
analyzing the feedback data from the user and responsive to the analyzing, generating and outputting an adjusted instruction state and an adjusted non-instruction state to the user, such that the adjusted instruction state comprises audio output, visual output, or both that prompts the user to take a second action or inaction and the first action is different than the second action.

2. The method of claim 1 wherein the first set of data is selected from one or more of the following: user account information, user preference, user selection, user input, user biometrics, user history, or auxiliary metadata; and the user input is received in text format, audio format, image format, or video format.

3. The method of claim 1 wherein the feedback data comprising one or more of the following: user input and user biometrics.

4. The method of claim 1 further comprising receiving a second set of data and using the second set of data to update the first set of data.

5. The method of claim 1 wherein the second set of data comprises one or more of the following: user feedback, user preferences, session results, and user evaluation of the session.

6. The method of claim 1 wherein analyzing and generating comprising comparing a user's current relaxation state in relation to a prior relation state and determining which instruction states or one or more non-instruction states increased the user's relaxation state and responsive to the determining, repeating the instruction states or the one or more non-instruction states which increased the user's relaxation level.

7. The method of claim 1 wherein a difference between the non-instruction state and the adjusted non-instruction state is the duration of the adjusted non-instruction state.

8. A meditation session generating system comprising:
a user interface configured to receive input and provide instructions to a user, such that the input comprises one or more of the following: user data, non-user data, and feedback data;
a processor configured to run machine executable code;
a memory storing non-transitory machine executable code, the machine executable code configured to:
process the user data and non-user data to generate a first instruction state and a first non-instruction states, an instruction state comprising audio output, visual output, or both that prompts the user to take a first action;
analyze the feedback data to perform one or more of the following:
repeat the first instruction state;
repeat the first non-instruction state;
adjust the first instruction state;
adjust the first non-instruction state; and
output one or more of the first instruction states, the first non-instruction states, the adjusted first instruction state, and the adjusted first non-instruction state to the user.

9. The system of claim 8, wherein the feedback data comprises one or more of the following: user input in text format, audio input, image input, video input, and user biometrics.

10. The system of claim 8, wherein adjust the first instruction state comprises one of the following: adjust the output volume of the output of the instruction state, and generate a second instruction state to prompt the user to take a second action or inaction.

11. The system of claim 8, wherein the first non-instruction state comprises one or more of the following: a duration of silence, an audio output, or a visual output.

12. The system of claim 8, wherein adjust the first non-instruction state comprises one or more of the following: adjusting the output volume of the first non-instruction state, adjusting the duration of the first non-instruction state, and adjusting the output provided to the user during the first non-instruction state.

13. The system of claim 8, wherein process the user data comprises determining one or more of the following: the user's relaxation state, the user's emotional state, and the user's physical state.

14. The system of claim 8, wherein analyze the feedback data comprises comparing the user's current body condition to the user's body condition at a prior point in time.

15. The system of claim 8, wherein the machine executable code comprises one or more algorithms used to process and analyze the user data and the feedback data, and the machine executable code further configured to use the feedback data to update the one or more algorithm to be executed during the meditation session.

16. A method for dynamically adjusting an output in a meditation session comprising: receiving a first set of data from the user, the first set of data indicating a first condition of the user;
processing the first set of data to generate a first instruction output;
providing the first meditation instruction output to the user;
receiving a second set of data from the user, the second set of data obtained from the user during the presentation of the first output and indicating a second condition of the user;
comparing the second set of data to the first set of data to determine whether the first instruction output improved the second condition of the user as compared to the first condition of the user; and
responsive to the comparing, either iterating the first instruction output, or presenting a second instruction output to the user to improve the meditation session for the user.

17. The method of claim 16 wherein the comparing determines whether the first instruction output increased relaxation of the user based on biometric data, and responsive to the first instruction output increasing relaxation of the user, repeating the first instruction output.

18. The method of claim 16 further comprising, responsive to the comparing, determining whether to terminate the meditation session.

19. The method of claim 16 wherein the first set of data is selected from one or more of the following: user account information, user preference data, user selection input, user input, user biometrics, user history, and auxiliary metadata.

20. The method of claim 16 wherein the second set of data is user input, user biometrics, or both.
